(19) United States
(12) Patent Application Publication
(10) Pub. No.: US 2026/0224786 A1
(43) Pub. Date: Aug. 6, 2026

(54) COLOSTRUM COLLECTOR

(71) Applicant: Fridababy, LLC, Miami, FL (US)

(72) Inventors: Chelsea HIRSCHHORN, Miami Beach, FL (US); Samuel SAUCEDA, Los Angeles, CA (US); Joseph MEDRANO, Miami, FL (US); John FRITZ, Orchard Park, NY (US)

(21) Appl. No.: 19/278,419

(22) Filed: Jul. 23, 2025

Related U.S. Application Data
(60) Provisional application No. 63/748,175, filed on Jan. 22, 2025.

(51) Int. Cl. A61M 1/06; A61J 9/00

(52) U.S. Cl. CPC A61M 1/062; A61J 9/00; A61M 2202/0014; A61M 2202/04

(57) ABSTRACT

A colostrum collection device includes a lower funnel-shaped component and an upper bulb component integrated with the lower funnel-shaped component. The colostrum collection device also includes a cap for sealing the lower funnel-shaped component. The colostrum collection device may be attachable to a T-shaped adapter.

### CROSS-REFERENCE TO RELATED APPLICATION

The present application claims the benefit of U.S. Provisional Patent Application No. 68/863,281, filed on Jan. 22, 2025, and titled “Colostrum Collector,” the disclosure of which is expressly incorporated by reference in its entirety.

### BACKGROUND

### Technical Field

Aspects of the present disclosure generally relate to a colostrum collector, and more specifically to a breast pump with a detachable colostrum collector.

### Description of the Related Technology

Colostrum is the first form of milk produced by humans during late pregnancy and in the initial days postpartum. Colostrum is characterized by its thick, yellowish appearance and has a high concentration of nutrients, antibodies, and growth factors. Although produced in small quantities, typically 2 to 20 milliliters per feeding, colostrum is rich in immunoglobulins, leukocytes, and antimicrobial peptides that help establish the infant's gut microbiota and strengthen immune defenses. Despite its limited volume, colostrum contains bioactive compounds that promote intestinal maturation and support overall neonatal health. Due to its viscosity and limited volume, colostrum is typically collected through manual hand expression, as its viscosity and small volume make mechanical pumping less effective. During expression, colostrum is released drop by drop from the nipple, and it is often collected into sterile containers or syringes for storage.

### SUMMARY

Various aspects of the present disclosure are directed to a colostrum collection device configured to facilitate efficient, hygienic expression, collection, and storage of colostrum. In one example, the device includes a lower funnel-shaped component designed to capture colostrum during manual or pump-assisted expression. This component tapers from a wide upper opening to a narrower section, guiding colostrum efficiently into a storage chamber while minimizing pooling and fluid loss. An upper bulb component may be integrated with the lower funnel-shaped component and may serve multiple functions, including assisting in the creation of suction during manual expression or enabling direct dispensing of colostrum for feeding. The integration of these components into a single unit reduces handling steps and potential contamination, thereby preserving the limited volume of colostrum typically produced. The device further includes a cap for sealing the lower funnel-shaped component, allowing the collected colostrum to be safely stored, refrigerated, or frozen. In some embodiments, the cap may be tethered to the device for ease of use and may include features for labeling or secure transport. Additionally, the colostrum collection device may be attachable to a T-shaped adapter, which allows it to interface with a manual or electric breast pump system. The adapter facilitates the separation of airflow and fluid flow, enabling colostrum to be directed efficiently into the collector while preserving suction. Overall, the device supports low-loss, hygienic collection of colostrum in both home and clinical settings.

Some aspects of the present disclosure are directed to a colostrum collection system including a breast pump, which may be manual or electric, and a flange configured to form a seal against the user's breast to facilitate expression. The system further includes a T-shaped adapter having a horizontal portion and a perpendicular vertical portion. The horizontal portion connects the flange to the pump, allowing airflow and suction to pass through uninterrupted, while the vertical portion is configured to receive and direct colostrum into a dedicated collection vessel. Attached to the vertical portion of the adapter is a colostrum collector, which comprises a lower funnel-shaped body for capturing and channeling colostrum, an upper bulb component that may assist with suction or direct feeding, and a tethered cap for sealing the collector after use. The funnel shape promotes efficient collection with minimal loss, while the integrated bulb allows the collector to function both as a storage vessel and a feeding tool. The tethered cap helps preserve sterility and prevents misplacement.

Some aspects are further directed to a method for collecting colostrum using a modular colostrum collection device and adapter system. The method begins by attaching a flange to one end of a horizontal portion of a T-shaped adapter, and attaching a breast pump (for example, manual, electric, or battery-powered) to the opposite end of the horizontal portion. A colostrum collector may be connected to the vertical portion of the adapter, forming a sealed and functional assembly. Once the components are connected, the flange is placed against the user's breast to create a secure seal. The breast pump is then activated, generating suction that facilitates expression. As colostrum is expressed from the breast, it travels through the flange and into the adapter. An internal diverter within the adapter redirects the colostrum through the vertical portion and into the collector, isolating the fluid path from the pump's primary suction channel.

Aspects generally include a method, apparatus, system, computer program product, non-transitory computer-readable medium, user equipment, base station, wireless communication device, and processing system as substantially described with reference to and as illustrated by the accompanying drawings and specification.

The foregoing has outlined rather broadly the features and technical advantages of examples according to the disclosure in order that the detailed description that follows may be better understood. Additional features and advantages will be described. The conception and specific examples disclosed may be readily utilized as a basis for modifying or designing other structures for carrying out the same purposes of the present disclosure. Such equivalent constructions do not depart from the scope of the appended claims. Characteristics of the concepts disclosed, both their organization and method of operation, together with associated advantages will be better understood from the following description when considered in connection with the accompanying Figures. Each of the Figures is provided for the purposes of illustration and description, and not as a definition of the limits of the claims.

### BRIEF DESCRIPTION OF THE DRAWINGS

So that features of the present disclosure can be understood in detail, a particular description may be had by reference to aspects, some of which are illustrated in the appended drawings. It is to be noted, however, that the appended drawings illustrate only certain aspects of this disclosure and are therefore not to be considered limiting of its scope, for the description may admit to other equally effective aspects. The same reference numbers in different drawings may identify the same or similar elements.

FIGS. 1A and 1B illustrate exploded views of an example colostrum collection system, in accordance with various aspects of the present disclosure

FIGS. 2A and 2B illustrate examples of a pump adapter, in accordance with various aspects of the present disclosure.

FIG. 2C illustrates a cross-sectional view of an example T-shaped adapter for use in a colostrum collection system, in accordance with various aspects of the present disclosure.

FIG. 2D illustrates a view of the adapter, showing the internal configuration of the adapter from the pump-side end, in accordance with various aspects of the present disclosure.

FIG. 2E provides a view of the adapter as seen from the flange-side opening, illustrating the internal configuration of the diverter and airflow structure, in accordance with various aspects of the present disclosure.

FIGS. 3A and 3B illustrate an example of the colostrum collection system, in accordance with various aspects of the present disclosure.

FIG. 4 illustrates a side view of an example colostrum collector, in accordance with various aspects of the present disclosure.

FIG. 5A illustrates an example embodiment of a colostrum collector equipped with a suction feeding tip, in accordance with various aspects of the present disclosure.

FIG. 5B illustrates an example of a colostrum collection system alongside a separately depicted suction feeding tip, in accordance with aspects of the present disclosure.

FIG. 6 illustrates a flow diagram for a process of using a colostrum collection system, in accordance with various aspects of the present disclosure

### DETAILED DESCRIPTION

The detailed description set forth below, in connection with the appended drawings, is intended as a description of various configurations and is not intended to represent the only configurations in which the concepts described herein may be practiced. The detailed description includes specific details for the purpose of providing a thorough understanding of the various concepts. It will be apparent to those skilled in the art, however, that these concepts may be practiced without these specific details. In some instances, well-known structures and components are shown in block diagram form in order to avoid obscuring such concepts.

Based on the teachings, one skilled in the art should appreciate that the scope of the present disclosure is intended to cover any aspect of the present disclosure, whether implemented independently of or combined with any other aspect of the present disclosure. For example, an apparatus may be implemented, or a method may be practiced using any number of the aspects set forth. In addition, the scope of the present disclosure is intended to cover such an apparatus or method practiced using other structure, functionality, or structure and functionality in addition to, or other than the various aspects of the present disclosure set forth. It should be understood that any aspect of the present disclosure may be embodied by one or more elements of a claim.

The word “exemplary” is used herein to mean “serving as an example, instance, or illustration.” Any aspect described herein as “exemplary” is not necessarily to be construed as preferred or advantageous over other aspects.

Although particular aspects are described herein, many variations and permutations of these aspects fall within the scope of the present disclosure. Although some benefits and advantages of the preferred aspects are mentioned, the scope of the present disclosure is not intended to be limited to particular benefits, uses, or objectives. Rather, aspects of the present disclosure are intended to be broadly applicable to different technologies, system configurations, networks, and protocols, some of which are illustrated by way of example in the Figures and in the following description of the preferred aspects. The detailed description and drawings are merely illustrative of the present disclosure rather than limiting, the scope of the present disclosure being defined by the appended claims and equivalents thereof.

As discussed, in humans, colostrum is produced in relatively small quantities, typically ranging from 2 to 20 milliliters per feeding. Despite its limited volume, colostrum is rich in immunoglobulins, leukocytes, and antimicrobial peptides that help to establish the infant's gut microbiota and strengthen their immune defenses. It also contains bioactive compounds that promote intestinal maturation and support overall neonatal health. Colostrum is typically collected through manual hand expression, as its viscosity and small volume make mechanical pumping less effective. During expression, colostrum is released drop by drop from the nipple, and it is often collected into sterile containers or syringes for storage.

Collecting colostrum during the days immediately before and after childbirth presents significant challenges, even though it is highly beneficial for newborns. Specifically, the collection may be difficult due to colostrum's thick, viscous consistency and the small quantities typically produced, which range from just a few drops to around 20 milliliters. These unique properties mean that even minor inefficiencies during expression can lead to significant loss, depriving the newborn of this valuable resource.

Once colostrum has been expressed, it is crucial to have a safe and hygienic way to store and preserve it. Proper storage solutions should protect the colostrum from contamination while accommodating its small volume. Freezing may be useful to preserve the colostrum for later use, but many existing storage containers are not optimized for this purpose, leading to inefficiencies or waste. After birth, an additional challenge arises: feeding the baby the collected colostrum. This process should minimize loss and ensure that the maximum amount of colostrum reaches the newborn, yet existing feeding tools often involve multiple transfer steps, which increase the risk of spillage or contamination.

Manual hand expression, the most common conventional method for collecting colostrum, can be difficult, labor-intensive, and inefficient. This difficulty underscores the need for alternative methods of colostrum expression that are simple, effective, and designed specifically to address the unique characteristics of colostrum. Furthermore, after collection, there is a need for an organized system to store and manage multiple filled containers. Without an effective organizational solution, caregivers may struggle to track and manage the collected colostrum, particularly in the busy postpartum period.

Various aspects of the present disclosure are directed to overcoming longstanding challenges in the collection, storage, and feeding of colostrum. These challenges include the thick and viscous nature of colostrum, its limited volume, the difficulty of manual expression, the inefficiencies of mechanical pumping, and the risk of contamination or loss during transfer between collection and feeding containers. To address these issues, various aspects of the present disclosure are directed to a modular colostrum collection system that facilitates direct collection, hygienic storage, and immediate feeding using the same container.

In some examples, the system includes a colostrum collector that includes an upper component and a lower component. The lower component is a funnel-shaped reservoir configured to efficiently capture individual drops of colostrum as they are expressed from the nipple. This component is shaped to minimize surface area and internal dead space, thereby reducing colostrum loss due to adherence or pooling. The funnel may taper into a narrow cylindrical base for concentrated storage and sealing.

The upper component is a bulb or dome-shaped element that may serve multiple purposes. In some examples, the bulb facilitates suction-assisted manual collection. For example, the lower portion of the bulb may be compressible, allowing the user to create negative pressure by squeezing and releasing it, thereby drawing colostrum upward into the collector through the upper opening. This gentle suction mechanism is particularly beneficial for collecting small volumes of colostrum, which may not flow freely under gravity alone. Additionally, or alternatively, the bulb functions as a feeding tool once collection is complete. It may be used as a squeezable reservoir to dispense colostrum directly into the infant's mouth or may be coupled with a soft-tipped feeding nipple or dropper for more controlled delivery. By integrating collection and feeding capabilities into a single component, the bulb eliminates the need for intermediate transfer into a separate container, thereby reducing handling, preserving volume, and minimizing the risk of contamination. This dual-purpose design streamlines the feeding process and improves hygiene, especially in neonatal and hospital environments where sterility and efficiency are critical.

In some implementations, the upper and lower components of the colostrum collector may interconnect using a secure mechanical interface, which may include, but is not limited to, an interference fit, snap-fit, bayonet-style twist lock, or threaded engagement. These connection mechanisms are selected to provide a reliable seal during use while allowing for easy assembly and disassembly by the caregiver. The interlocking design may prevent or reduce leakage during collection, storage, or transport, while also supporting repeatable use and thorough cleaning between sessions. Alternatively, the upper and lower components may be integrally formed as a single unit, for example, through a molding process.

To further support hygienic handling and preservation, a tethered cap may be integrated with the lower portion of the collector. The cap may seal the outlet of the storage chamber after colostrum has been expressed, thereby creating a barrier against airborne contaminants and accidental spillage. The tethered configuration ensures that the cap remains attached to the device, reducing the risk of misplacement during use in busy postpartum or hospital environments.

In some examples, the cap is constructed of flexible, food-grade material that conforms to the shape of the container opening to maintain an airtight seal. As such, the colostrum collector may be well-suited for both short-term refrigeration and longer-term freezing. The cap may also include a tab, rib, or other ergonomic features to facilitate easy removal by the caregiver without compromising sterility. Taken together, these elements enhance the overall usability, safety, and cleanliness of the colostrum collection process, making it practical for both home and clinical use.

By integrating collection, storage, and feeding into a single streamlined device, the colostrum collector improves usability for caregivers, particularly in hospital or postpartum settings where time and cleanliness are critical. It also ensures that colostrum can be captured efficiently, even in low volumes, and delivered to the infant with minimal loss, helping to maximize the immunological and nutritional benefits during the earliest and most critical stages of development.

In some aspects, an adapter may be used to interface the colostrum collector with a breast pump, such as a manual, electric, or battery-powered pump. This may be positioned between a flange, which contacts the user's breast, and the main pump body. In some examples, the main pump body includes a valve and a milk collection chamber. The adapter provides a dedicated channel that redirects colostrum into a separate, colostrum collector before it reaches the pump's standard flow path or collection bottle. This configuration ensures that even the smallest amounts of colostrum, which might otherwise be lost in tubing or trapped behind valves, are efficiently captured and preserved.

Because colostrum is produced in limited quantities and is highly viscous, passing it through traditional pump valves and components can lead to adherence, retention, or dilution, resulting in significant loss. By bypassing these internal pump components, the adapter design minimizes these risks and improves recovery of expressed colostrum. The adapter may incorporate features such as a tapered port for a secure press-fit connection to the collector, flow diverters to guide liquid with minimal turbulence, and ribbed surfaces or gaskets to maintain airtight seals and preserve suction efficiency.

After expression, the colostrum collector may be detached from the adapter and sealed with a cap for hygienic storage. The sealed collector may be labeled with collection time and date and stored under refrigeration or placed in a freezer for later use. Because the adapter allows colostrum to be collected without additional transfer steps, the adapter reduces handling, lowers the risk of contamination, and helps caregivers retain the full nutritional and immunological value of this early milk. This design is particularly beneficial for hospital settings, neonatal intensive care units (NICUs), or home environments where precise collection and hygienic handling are desirable.

Accordingly, various aspects of the present disclosure are directed to a colostrum collector that addresses the conventional challenges of colostrum collection, storage, and feeding. By integrating features that address each stage of the process (e.g., manual or pump-assisted expression, safe storage, and direct feeding), various aspects of the present disclosure ensure that colostrum is preserved and delivered to the baby efficiently, hygienically, and with minimal loss. Additionally, the colostrum collector provides a user-friendly and practical approach for caregivers, improving the overall experience of colostrum collection and feeding during the critical postpartum period.

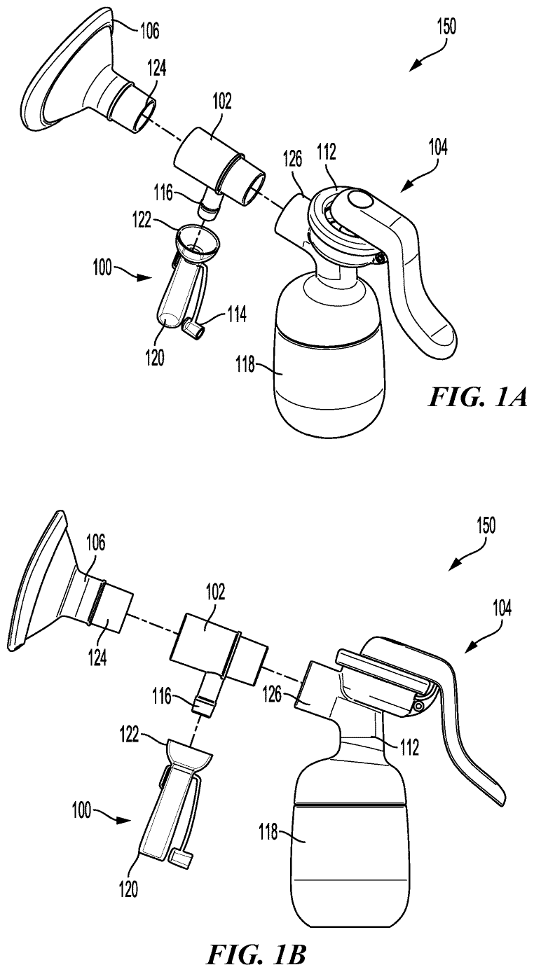

FIGS. 1A and 1B illustrate exploded views of an example colostrum collection system 150, in accordance with various aspects of the present disclosure. The colostrum collection system 150 may be specified for use with a breast pump assembly. The system 150 includes a colostrum collector 100, a T-shaped pump adapter 102, a breast pump 104, and a breast flange 106. As shown, the pump adapter 102 is positioned between the flange 106 and the inlet of the breast pump 104, forming a modular and detachable assembly. The adapter 102 facilitates the diversion of the colostrum away from the primary pump body and into the colostrum collector 100. In the examples shown, the breast pump 104 is a manually actuated device that uses a lever to generate negative pressure. However, aspects of the present disclosure are not limited to manual pumps. In alternate examples, the adapter 102 may be used with electric, battery-powered, or hospital-grade breast pumps, provided that the flange 106 and pump body 104 accept a compatible connection interface 102. In still other examples, the adapter 102 may be integrated directly into the flange 106 or a housing 112 of the pump 104 to reduce the number of discrete components. The housing 112 may also be referred to as a body of the pump 104 or the pump body (hereinafter used interchangeably).

The breast pump 104 may include a milk storage unit 118 configured to receive and store milk expressed from the breast during operation of the pump 104. The milk storage unit 118 may be removably attached to the pump body 112 and may comprise a bottle, container, bag, or other receptacle suitable for the temporary or long-term storage of expressed breast milk. In some examples, the milk storage unit 118 is formed from a sterilizable, BPA-free material and may include volume markings, anti-spill features, or insulation for thermal regulation. Although shown as part of a manual breast pump 104, the storage unit 118 may be adapted for use with electric or hospital-grade pumps, and may be compatible with a variety of standardized bottle neck geometries or proprietary interfaces. In some examples, the milk storage unit 118 may include a one-way valve or backflow protector to prevent contamination of upstream components during pumping.

The flange 106 may be shaped to conform to the user's breast and may be made from soft, biocompatible material (e.g., silicone or thermoplastic elastomer) to form a seal that maintains suction during expression. The flange 106 channels the colostrum toward the adapter 102, which functions as a diverter to route the colostrum into the collector 100. The adapter 102 may include one or more internal passages, valves, or baffles to direct colostrum flow and maintain consistent suction. In some examples, the adapter 102 includes a bottom-facing port 116 configured to receive the colostrum collector 100. The port 116 may be tapered, threaded, or configured with a quick-connect or bayonet-style locking interface to facilitate secure engagement. In some examples, the distal end of the vertical port 116 has an outer diameter of approximately 7.5 mm. This diameter may be larger than the inlet diameter of standard syringes, which typically range from 4.0 to 4.5 mm. Of course, other diameters may be used.

While the adapter 102 is illustrated as having a generally T-shaped configuration, aspects of the present disclosure are not limited to this form. Other geometries, such as Y-shaped, elbow-shaped, or offset L-shaped adapters, may also be used to achieve the described functionality of separating airflow and fluid flow, directing colostrum into a collector while preserving suction through the primary air path.

As shown in FIGS. 1A and 1B, the adapter 102 may interface between the flange 106 and a body 112 of the breast pump 104. One end of the adapter 102 is designed to receive and connect to an outlet 124 of the flange 106, while the opposite end is configured to attach to and inlet 126 of the pump body 112, thereby forming a fluid path between the user's breast and the pump 104. The adapter 102 may provide a secure connection using an interference fit, snap-lock mechanism, threaded engagement, or other suitable coupling structure to ensure airtight and leak-proof operation during use.

In some examples, the adapter 102 may have a tapered design in which the end that connects to the outlet 124 has a larger diameter than the end that connects to the inlet 126. This tapered configuration accommodates differences in diameter between the flange outlet 124 and the pump inlet 126 and helps maintain a smooth, directed flow of colostrum or milk with minimal turbulence or fluid loss. The tapering may be continuous or stepped, and may optionally include external or internal ribs, ridges, or grooves to enhance grip and sealing. In other examples, the adapter 102 may be integrally formed with the flange 106 or the pump body 112, or may include flexible or articulated joints to allow angular adjustment of the flow path. This flexibility supports compatibility with a variety of pump geometries and user preferences, while ensuring efficient and hygienic routing of colostrum into the collector 100.

The colostrum collector 100 may receive and store expressed colostrum in a hygienic, low-loss manner. In some examples, the collector includes a cap 114 that is tethered to the lower body 120 of the collector 100 to ensure that the cap 114 is not misplaced and remains available after use. The cap 144 may seal the collector 100 after expression to preserve sterility and prevent leakage during handling or storage. In some examples, the cap 114 may include an integrated labeling surface, tamper-evident features, or a snap-in vent to facilitate cooling. Although the example shown includes a tethered cap 114, alternate sealing mechanisms may include twist-off lids, snap-fit plugs, foil seals, or resealable membranes. In still other examples, the collector 100 may be configured with a septum or self-sealing port for needle-free extraction or direct syringe interface.

The collector 100 may be constructed from medical-grade materials such as polypropylene, polyethylene, or other biocompatible plastics that are approved for contact with human milk and suitable for both refrigeration and freezing. These materials may be selected for their durability, chemical resistance, and ability to maintain structural integrity under sterilization procedures such as autoclaving, boiling, or UV treatment. In some examples, one or more components of the collector 100, such as the upper bulb portion 122 or lower portion 120, may be made from silicone or another flexible elastomer. The elasticity and resilience of silicone also make it well-suited for direct feeding applications, as it can be used to gently dispense colostrum through controlled squeezing.

In some examples, different materials may be used for different sections of the collector 100 to improve functionality. For example, the funnel portion 120 may be rigid for structural stability, while the bulb portion 122 may be soft and pliable. In some cases, the collector 100 may include multi-layered constructions or coatings, such as hydrophobic inner linings to reduce colostrum adhesion, or antimicrobial surface treatments to enhance hygiene. The selection of materials and configurations may vary depending on intended use, cost considerations, and user preferences, and various aspects of the present disclosure are not limited to any specific combination of materials described herein.

As shown in FIGS. 1A and 1B, the colostrum collector 100 may include two primary sections: a lower funnel-shaped component 120 and an upper bulb component 122. The lower component 120 includes a wide opening for dropwise capture and tapers into a cylindrical or conical section for efficient flow into a storage chamber. The geometry is designed to reduce pooling, minimize surface retention, and guide even small amounts of colostrum into the main reservoir. In some examples, the lower component 120 may include internal hydrophobic or non-stick coatings to prevent loss through adhesion. In some examples, the lower component 120 may be collapsible or flexible to accommodate compact storage or portability. Additionally, in some examples, the lower portion 120 may be integrated with volume markings, fill indicators, or ridges to assist in measurement and handling.

The upper component 122 may be a bulb-shaped structure that serves multiple purposes. In some examples, the upper component 122 functions as a manually squeezable suction bulb to assist with colostrum extraction during hand expression. When squeezed and released, the upper component 122 can generate negative pressure sufficient to draw colostrum into the lower portion 120. In other configurations, the upper component 122 may serve as a feeding aid, enabling direct delivery of colostrum to the infant either by dispensing through a soft-tipped nozzle or by attachment to a feeding nipple or oral syringe. The upper component 122 may be removable and re-attachable using a snap-fit, thread-on, or bayonet connection, allowing for flexible use as either a collection or feeding module. In some examples, the upper component 122 may incorporate a one-way valve to control dispensing rate, a self-venting system to equalize pressure, or a filter element to ensure sterility. The modular nature of the collector 100 allows it to function as a standalone container, as part of a breast pump assembly, or as a delivery system, reducing handling steps, contamination risk, and material waste. Additionally, the modular components may be interchangeable, allowing the user to switch between different bulb styles, cap types, or storage configurations depending on the use case or clinical requirements.

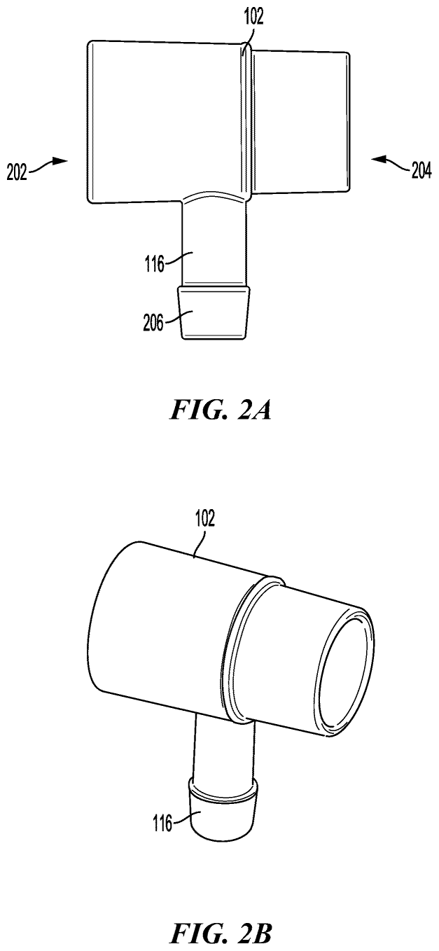

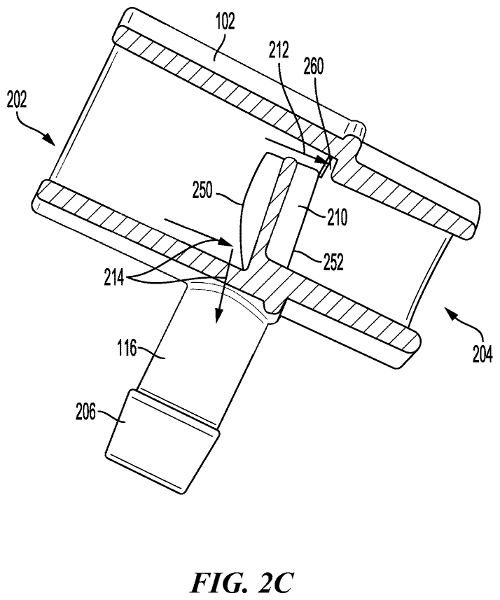

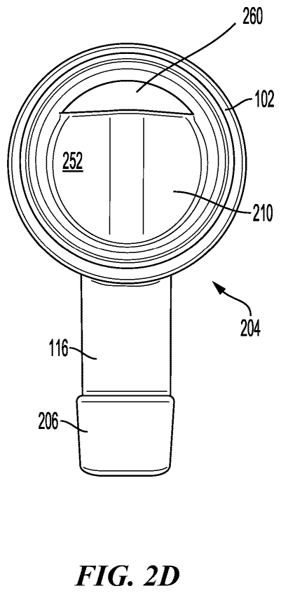

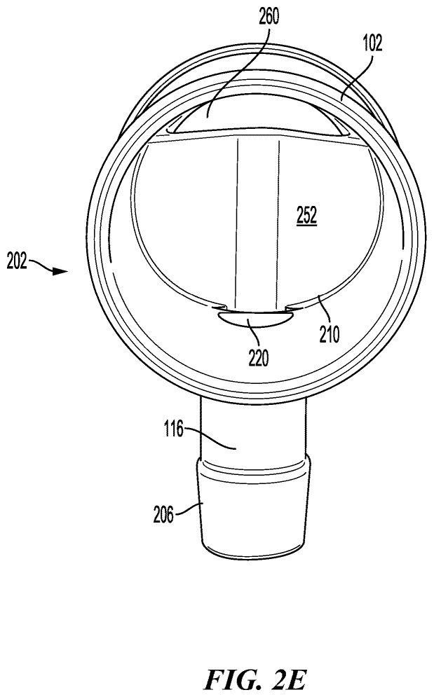

FIGS. 2A and 2B illustrate examples of a pump adapter 102, in accordance with various aspects of the present disclosure. The pump adapter 102, also referred to as an adapter, may serve as an interface between a breast pump assembly and a colostrum collector. The adapter 102 may be a generally T-shaped connector comprising a horizontally extending section and a downwardly extending vertical port 116. As shown in FIG. 2A, the horizontal section includes a first circular opening 202 at one end, which is configured to receive a breast pump flange, and a second circular opening 204 at the opposite end, which is configured to connect to the pump body. These openings may be aligned along a common axis to permit direct airflow and suction transmission between the flange and the pump.

In some examples, the horizontal section may be tapered from the flange-side opening 202 toward the pump-side opening 204. This tapering allows the adapter 102 to accommodate flange and pump components with different diameters, ensuring a snug fit on both ends. For example, the flange-side opening 202 may be wider than the pump-side opening 204 to accommodate the flange's larger outlet and the pump's narrower inlet. The taper may be internal, external, or both, and may be gradual or stepped. The vertical port 116 projects perpendicularly from the central portion of the horizontal section and is configured to receive and direct the colostrum into a colostrum collector. In some examples, the port 116 includes a tapered distal end 206 or may incorporate external ribs or grooves to provide an interference or snap-fit with the collector, ensuring a secure and leak-resistant attachment during use.

The interior of adapter 102 defines a channel system that connects openings 202 and 204 of the horizontal section, as well as a channel from the flange-side opening 202 to the vertical port 116. This configuration enables colostrum expressed through the flange to flow efficiently into the collector via the port 116. That is, the adapter 102 may include internal flow guides or diverter structures to direct the colostrum toward port 116 under low-pressure suction.

The outer surfaces of the adapter 102 may include annular rims, raised rings, or gripping features to enhance compatibility with mating components and to support user assembly. For example, outer surfaces near the opening 204 may feature alignment ribs or threading to interface with the pump body, while the flange-facing end at opening 202 may include grooves to retain a sealing gasket or O-ring. The adapter 102 may be integrally molded as a unitary component from medical-grade polymers such as polypropylene or polycarbonate, or it may be formed as a multi-part assembly to allow for reconfiguration or cleaning. While FIGS. 2A and 2B depict a T-shaped adapter, alternative embodiments may employ angled ports, swivel joints, or modular inserts to accommodate different pump geometries, flow paths, or collector positions.

FIG. 2C illustrates a cross-sectional view of an example T-shaped adapter 102 for use in a colostrum collection system, in accordance with various aspects of the present disclosure. The adapter 102 may separate airflow and colostrum flow during breast milk expression, thereby improving the efficiency and hygiene of colostrum collection. The adapter 102 includes a horizontal portion with two opposing ends: a first end 202 configured to connect to a breast flange, and a second end 204 configured to connect to a breast pump. A vertical port 116 extends downward from the intersection of the horizontal portion and terminates in a tapered outlet 206 configured to interface with a colostrum collector.

Positioned within the interior of the adapter 102 is a diverter 210, which, in some examples, is formed as a curved, funnel-like block structure. Specifically, a portion 250 of the diverter 210 facing the first opening 202 may be curved, and another portion 252 facing the second opening 204 may be flat. The diverter 210 may be oriented at a non-perpendicular angle relative to the vertical port 116, allowing colostrum to be smoothly redirected from the horizontal flow path into the downward-facing port 116, as shown by arrows 214. This angled, contoured geometry promotes laminar flow and reduces the likelihood of turbulence, pooling, or retention of colostrum inside the adapter. In contrast, airflow from the breast pump is directed straight through the horizontal passage 260 of the adapter, as indicated by arrow 212, enabling continuous suction while keeping colostrum isolated from the main pump body.

In some examples, the distal end 206 of the vertical port 116 may be inserted into the inlet of the colostrum collector, as opposed to the collector being inserted into the adapter. This design facilitates more effective drainage of colostrum into the collector and minimizes the risk of fluid pooling or clogging at the collector entry point. In one example, the outer diameter of the distal end 206 is approximately 7.5 millimeters. However, aspects of the present disclosure are not limited to this dimension, and other diameters, geometries, and coupling configurations are contemplated to accommodate various collector types, sizes, and application-specific requirements.

By optimizing both the internal diverter geometry and the physical interface between the adapter and the collector, the design shown in FIG. 2C improves the directionality of flow, reduces handling loss, and enhances the overall efficiency and usability of the colostrum collection system.

FIG. 2D illustrates a view of the adapter 102, showing the internal configuration of the adapter from the pump-side end 204, in accordance with various aspects of the present disclosure. Prominently featured in this view is the diverter 210, which is positioned within the internal flow path to guide colostrum into the vertical port 116. Surrounding the diverter is a horizontal opening 260, which defines the main airflow passage extending through the adapter from the pump-side opening 204 toward the flange-side opening 202 (not shown in this view).

The horizontal opening 260 is generally annular or ring-shaped, forming a wide circular passage that circumferentially surrounds the upper portion of the diverter 210. This ring-like geometry expands the available surface area through which air can flow, enabling continuous and unobstructed suction transmission between the pump and the flange, even while the diverter redirects colostrum downward. The open geometry of the channel 260 may prevent the diverter 210 from blocking airflow. That is, the channel 260 allows air to pass around and over the diverter 260 through the annular gap.

The broad profile of the opening 260 contributes to increased airflow efficiency by reducing turbulence and minimizing pressure drop across the adapter. Unlike a narrow or obstructed passage, the wide circumferential nature of opening 260 (e.g., across a width between inner walls) enables more uniform and distributed airflow, supporting consistent negative pressure during breast pump operation. This configuration ensures that suction is maintained throughout the pumping session while allowing colostrum to be diverted separately into the vertical port 116, thereby optimizing both fluid and air handling within the adapter.

FIG. 2E provides a view of the adapter 102 as seen from the flange-side opening 202, illustrating the internal configuration of the diverter and airflow structure, in accordance with various aspects of the present disclosure. From this perspective, several components are visible, including the internal diverter 210, the colostrum flow channel 220, and the surrounding horizontal opening 260.

At the center of the adapter interior is the diverter 210, which is shaped as a curved, funnel-like surface 252 that slopes downward toward the vertical port 116. This diverter defines the boundary of a colostrum collection region and includes a central flow channel 220 formed at the lower end of the diverter surface. The flow channel 220 serves as a pathway that guides colostrum expressed from the flange down into the vertical port 116, and ultimately into a colostrum collector. The diverter 210 may be oriented at a non-perpendicular, oblique angle relative to the vertical port 116. This geometry facilitates laminar redirection of colostrum into the collector and reduces turbulence. The curve and angle of the diverter 210 enable smoother flow transitions than would be achievable with flat or orthogonally positioned diverter blocks, which tend to cause backflow or droplet adhesion.

Surrounding the diverter is the horizontal opening 260, which is shown in FIG. 2E as a generally annular or ring-shaped gap that is above the central diverter 210. This opening allows air, drawn in by the breast pump, to flow freely across the interior surface of the adapter and continue through the adapter's main channel. The broad, open shape of the channel 260 promotes efficient and uniform suction, ensuring that negative pressure from the breast pump is evenly distributed while colostrum is simultaneously diverted downward through the channel 220.

The view of FIG. 2E shows how the geometry of the diverter 210 and flow channel 220 cooperate to isolate and direct colostrum into the vertical outlet, while the surrounding opening 260 ensures unrestricted air passage. This separation of airflow and colostrum flow enhances suction stability, minimizes fluid loss, and supports hygienic, low-turbulence routing of colostrum to the collector.

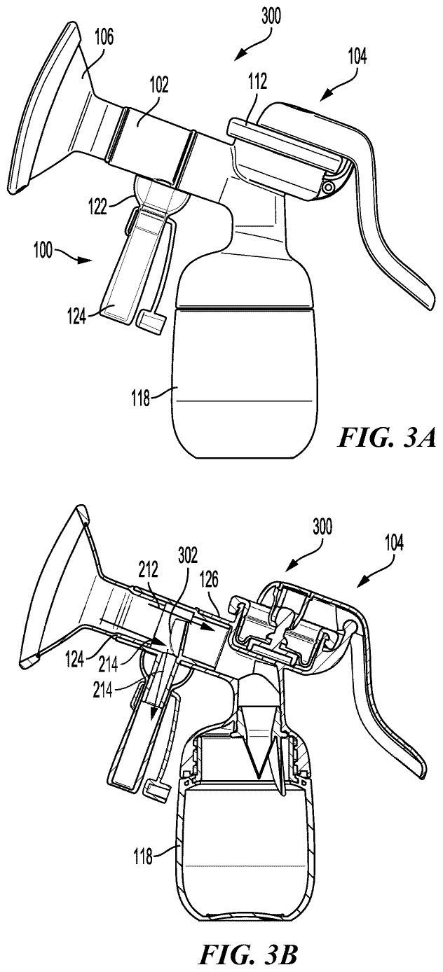

FIGS. 3A and 3B illustrate an example of the colostrum collection system 300, in accordance with various aspects of the present disclosure. As shown in the examples of FIGS. 3A and 3B, the pump adapter 102 is interposed between the flange 106 and the breast pump 104, and the colostrum collector 100 is attached at the lower part of the adapter. For clarity, many of the individual components shown in FIGS. 3A and 3B, including the flange 106, pump adapter 102, collector 100, and milk storage unit 118, have been described in detail with reference to FIGS. 1A, 1B, 2A, and 2B, and such descriptions are omitted here for brevity. Where possible, elements are labeled consistently across the Figures to enhance understanding of how the components interact within the system.

As shown in FIG. 3A, the system 300 is assembled such that the flange 106 is securely attached to the first opening 202 of the adapter 102, while the opposite opening 204 is coupled to the pump body 112 of the breast pump 104. The vertical port 116 of the adapter 102 receives the upper portion 122 of the colostrum collector 100, forming a sealed, downward-facing channel for colostrum capture. When a user manually operates the handle of the pump 104, negative pressure is generated within the flange 106, which draws colostrum expressed from the breast through the adapter 102 and into the collector 100. The adapter 102 thus functions to separate the colostrum flow from the main milk path of the breast pump 104 while preserving suction continuity.

In the cross-sectional view of FIG. 3B, additional internal features of the system are visible. The adapter 102 may optionally include an internal diverter 302 that directs colostrum flow toward the vertical port 116 and into the collector 100 via a secondary flow channel 306. A primary flow channel 304 may be present to permit the continuous passage of air during operation; however, the system is specifically designed such that fluid, including milk or colostrum, does not flow through the primary channel 304. The adapter 102 may be used for colostrum collection, which typically involves small volumes that are to be captured entirely within the collector 100. When mature milk expression is required, the adapter may be removed, and milk may be collected through standard pump operation without the diverter in place. In some examples, the primary flow channel 304 may incorporate a flow control mechanism, such as a flap valve, check valve, or pressure-sensitive membrane, configured to allow air to pass while preventing the passage of liquid. This configuration ensures that suction is preserved while maintaining a sealed, hygienic fluid path for colostrum diversion and collection.

In alternative configurations, the internal channel of the adapter 102 may be entirely hollow, relying solely on the force of gravity and applied suction to guide fluid flow into the colostrum collector or milk bottle. In yet other examples, the primary channel 304 may serve as an air passage rather than a liquid flow path. For instance, the breast pump 104 may draw air through the primary channel 304 via the pump mechanism, thereby creating a vacuum effect that assists in pulling the colostrum through the flange 106 and into the collector 100. This configuration may be advantageous when the adapter 102 is configured solely for colostrum collection and breast milk collection is not needed.

The system may also be configured to allow modular reconfiguration. In some examples, the colostrum collector 100 may be removed and replaced with a valve cap or plug when colostrum collection is not required. Conversely, the milk collection container 118 may be detached when the device is used exclusively for colostrum expression, minimizing the risk of contamination and simplifying cleaning. Furthermore, the diverter 302 may be removable for cleaning or replacement, or may be replaced with diverters of varying geometries or materials (e.g., silicone vs. rigid polymer) to tailor the fluid dynamics for different user preferences or fluid viscosities.

While the aspects shown include a manual pump, the described system may be adapted for use with electric or hospital-grade breast pumps. In such configurations, the adapter 102 and diverter 302 may be adapted to accommodate higher or pulsed vacuum cycles, and may include reinforcement features such as ribs, seals, or locking rings to ensure stability and fluid-tight operation under dynamic pressure conditions.

In some examples, the colostrum collection system, such as the system 300 described with reference to FIGS. 3A and 3B, may include one or more integrated sensors to improve ease of use, safety, and data tracking. For example, a flow sensor may be embedded in an adapter, such as the adapter 102 described with references to FIGS. 1A, 1B, 2A, 2B, 3A, and 3B, or within the colostrum collector, such as the colostrum collector 100 described with references to FIGS. 1A, 1B, 3A, and 3B, to detect when colostrum is entering the device. This sensor may activate an indicator light or send a signal to an external device, such as a smartphone or hospital monitor, when flow begins, stops, or exceeds a defined threshold. The system may also include a visual or digital colostrum volume indicator, which could be implemented using an optical sensor, capacitive sensor, or graduated markings visible on the exterior of the collector.

In other examples, a temperature sensor may be included in the collector or near the point of expression to monitor the temperature of the collected colostrum. If the temperature rises above or drops below a safe threshold, the system may issue a visual or audible alert or log the event for later review. This may be desirable when collected colostrum must be handled according to strict medical or storage guidelines, such as in hospital neonatal units or donor milk programs.

Some systems may include wireless communication components such as Bluetooth or Wi-Fi modules. These may allow the colostrum collection system to transmit session data, including duration, volume, temperature, and flow patterns, to a companion mobile app or centralized hospital monitoring system. For example, a user may open an app that displays real-time feedback on suction strength and estimated collected volume, or receive a notification when colostrum flow stops. In another case, a clinician may review logs for multiple sessions to evaluate how much colostrum was expressed in the first 24 hours after delivery, which can be a critical metric for newborn care.

In some examples, the adapter may include a smart diverter that changes the fluid path based on sensor input. For instance, the diverter may route initial low-volume, high-viscosity colostrum to the collector, and later route mature milk to a standard milk bottle as flow rate increases. This diverter may be mechanically actuated by a solenoid or motor, or may use a passive system such as a pressure-responsive flap. The diverter may also be controlled electronically using feedback from flow or viscosity sensors, allowing dynamic switching without user intervention. This may be desirable for mothers who begin collecting colostrum and then transition into full milk expression during the same session.

The collector itself may include additional features to enhance functionality. For example, it may be constructed of transparent or semi-transparent material with printed volume indicators for easy visual measurement. A tethered cap may include a writable label area or a scannable QR code for tracking collection time, user ID, or storage instructions. Some collectors may be single-use for sterile applications, while others may be reusable and dishwasher-safe for home use. In systems designed for bilateral use, two collectors may operate simultaneously with synchronized sensors and dual tracking on a connected app, allowing independent monitoring of left and right expression.

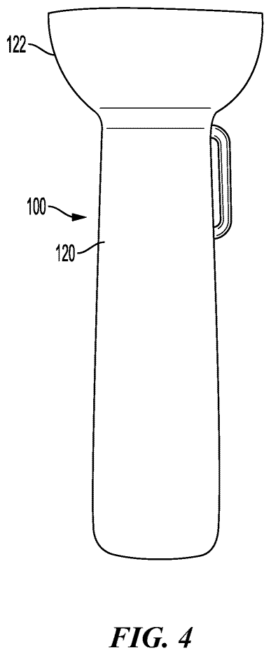

FIG. 4 illustrates a side view of an example colostrum collector 100, in accordance with various aspects of the present disclosure. The colostrum collector is shown in an assembled configuration, consisting primarily of two components: a lower funnel-shaped body 120 and an upper bulb component 122.

The lower portion 120 serves as the main collection and storage chamber for expressed colostrum. It is generally cylindrical or conical in shape, narrowing slightly toward the base, which may be flat or rounded. This shape promotes ease of handling, stable storage, and efficient transfer of colostrum into the collector when used with either manual hand expression or a pump-adapter interface. The lower portion may also be constructed from a flexible, medical-grade material, such as silicone or polypropylene, to accommodate compressive suction or allow for gentle squeezing during feeding or cleaning.

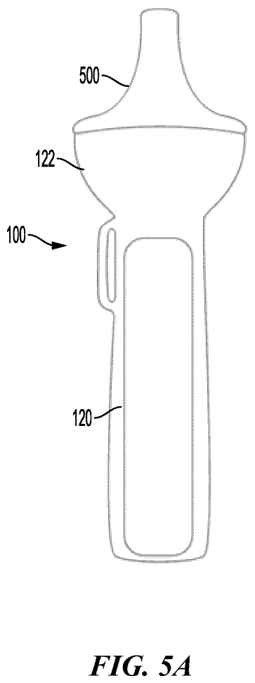

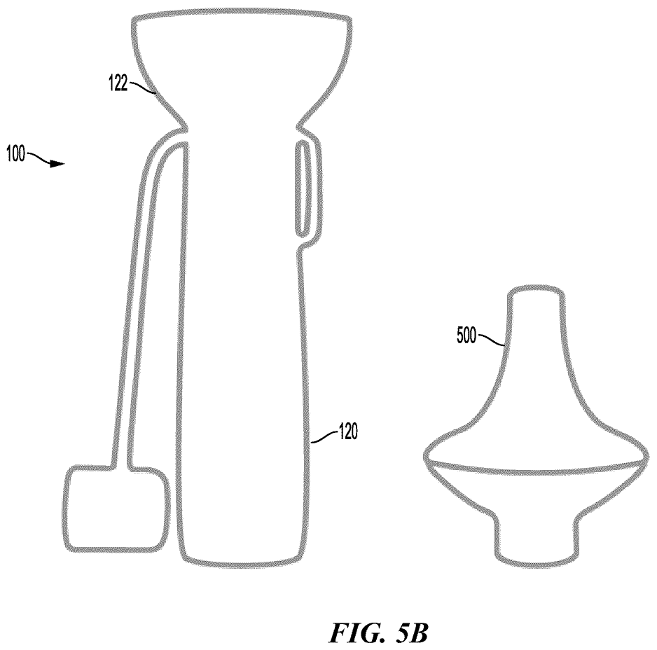

The upper portion 122 may be fixed or removably attached to the lower portion and has a wider, bowl- or dome-like shape. This upper component may serve several functions depending on the specific configuration. In some embodiments, it may be used to assist in drawing colostrum into the collector via compression and suction. In others, it may function as an attachment point for a feeding element, such as a suction tip or nipple (as shown in FIG. 5). The wide upper opening facilitates dropwise collection and may also support convenient cleaning or manual inspection of the contents.

Together, components 120 and 122 form an integrated and ergonomic colostrum collection device 100 that can be used for both expression and feeding. FIG. 4 emphasizes the clean, functional geometry of the device and its ability to transition from collection to feeding without requiring additional containers or complex handling steps.

FIG. 5A illustrates an example embodiment of a colostrum collector 100 equipped with a suction feeding tip 500, in accordance with various aspects of the present disclosure. The suction feeding tip 500 may enable direct administration of collected colostrum to an infant. The collector 100 includes two primary body components: a lower portion 120 and an upper portion 122. The lower portion 120 is generally cylindrical or tubular and serves as the main collection reservoir for storing expressed colostrum. This portion may be formed of a transparent or semi-transparent medical-grade material and may include volume markings, label areas, or tethered caps (not shown here) for sealing and tracking purposes.

The upper portion 122 may be integrated with the lower portion 120 and may be configured to interface with a feeding or suction accessory. In this example, the upper portion is fitted with a suction feeding tip 500, which extends upward in a tapered or nipple-like shape. The suction feeding tip 500 is designed to allow a caregiver to gently dispense colostrum directly into an infant's mouth using controlled squeezing of the collector body or natural infant suction. The tip 500 may be made of a soft, biocompatible material such as silicone to facilitate safe and comfortable feeding.

This configuration allows the colostrum collector 100 to transition from a passive storage device into an active feeding device without requiring colostrum to be transferred into a separate syringe, spoon, or bottle. By minimizing handling and transfer steps, the integrated feeding tip design helps preserve colostrum volume, reduce contamination risk, and streamline the feeding process. As shown in FIG. 5A, the collector with the suction feeding tip 500, offers a compact, ergonomic, and hygienic solution suitable for both home and clinical use.

FIG. 5B illustrates an example of a colostrum collection system 100 alongside a separately depicted suction feeding tip 500, in accordance with aspects of the present disclosure. On the left side of the figure is the colostrum collector 100, which includes a lower body portion 120 and an upper funnel-shaped portion 122. Also shown attached to the collector 100 is a tethered cap, which may be used to seal the lower body 120 for storage after collection. The funnel-shaped upper portion 122 is designed to facilitate dropwise collection of colostrum and may also serve as an attachment point for feeding accessories.

To the right of the figure is the suction feeding tip 500, which is shown detached from the collector. The tip 500 features a distinct hourglass or bell-shaped profile with a narrow outlet at the top, a widened midsection, and a reduced lower neck for attachment. This shape is ergonomically designed to support gentle, controlled feeding of colostrum to an infant. The narrow top may function as a mouthpiece or nipple-like extension that fits comfortably in an infant's mouth, while the broader central body allows the tip to be easily gripped or compressed by a caregiver for manual dispensing. The lower section of the tip is designed to connect securely to the upper portion 122 of the colostrum collector.

The bulbous, tapered geometry of the suction tip 500 enhances usability by enabling the caregiver to apply gentle pressure and precisely control the flow of colostrum. The shape may also help reduce pooling of liquid within the tip and improve overall feeding efficiency. In some embodiments, the suction tip may be made of a soft, flexible material such as silicone to ensure safety and comfort during use. FIG. 5B emphasizes the modular nature of the system, allowing caregivers to transition seamlessly from collection to direct feeding using the same colostrum container with minimal handling.

As discussed, various aspects of the present disclosure are directed to a colostrum collection device designed to improve the efficiency, hygiene, and usability of colostrum expression, particularly in the early postpartum period when small volumes of nutrient-rich colostrum are produced. The colostrum collection device includes a lower funnel-shaped component for receiving colostrum during expression and an upper bulb component that is removably attached to the funnel. This upper component may also serve as a feeding mechanism, enabling direct delivery of colostrum to an infant without requiring transfer to a separate container. A tethered cap is provided to seal the collection device for hygienic storage or freezing once collection is complete.

The colostrum collection device may interface with a T-shaped adapter, which facilitates integration with breast pump systems. The adapter includes a horizontal portion having a first opening for attaching to a breast flange and a second, typically narrower opening for attaching to a breast pump. The vertical portion of the adapter, which is perpendicular to the horizontal portion, connects to the funnel-shaped component of the collection device. This vertical portion may have a tapered end to ensure a secure and leak-resistant connection. In some examples, the adapter includes a diverter that channels colostrum specifically into the vertical port, enhancing collection efficiency and preventing the colostrum from being lost in the pump system. Taken together, the claimed system enables direct, low-loss collection of colostrum, seamless integration with existing pump hardware, and simplified feeding and storage, thereby addressing common pain points associated with early-stage milk expression.

The adapter may include an internal divider which functions as a curved, funnel-like diverter that guides colostrum efficiently into the channel leading to the collector. The distal end of the adapter that inserts into the collector has a diameter, for example, approximately 7.5 mm, which may be larger than a standard syringe. In some examples, the adapter is inserted into the collector, rather than the collector being inserted into an opening of a bottom-facing port of the adapter. This orientation improves colostrum flow into the collector and reduces the risk of fluid pooling or getting trapped at the entry point, thereby minimizing loss of this high-value substance. The adapter may include a broad, annular horizontal opening adjacent to the diverter, which forms a continuous air path for suction transmission between the pump and flange. This structure is distinguishable from conventional adapters that include narrow ducts or offset passages. By maximizing the cross-sectional area available for airflow, the annular design reduces turbulence, preserves suction efficiency, and ensures the negative pressure remains uninterrupted, even during simultaneous fluid diversion.

A process for collecting colostrum using the disclosed system may begin by providing a pump assembly that includes a breast flange, a T-shaped adapter, and a colostrum collector. The flange is connected to one end of the adapter. The opposite end of the adapter is connected to a breast pump. The collector is then attached to the downward-facing port of the adapter. The user places the flange against the breast to form a seal. The user activates the breast pump to generate suction. As suction is applied, colostrum is expressed from the breast. The colostrum flows from the flange into the adapter. The colostrum is then directed into the collector through the vertical port. A flow sensor within the system may detect the presence of colostrum. Upon detection, the sensor may trigger a signal, such as an LED indicator or a wireless notification sent to a mobile application. A temperature sensor may simultaneously monitor and log the temperature of the collected colostrum.

The process may further include viewing real-time data on a connected display, such as a mobile device or monitoring station. This data may include session duration, flow rate, or estimated volume collected. Once the user determines that collection is complete, the collector is removed from the adapter. The user seals the collector using a tethered cap or similar closure. The sealed collector is then placed in a refrigerated or frozen environment for storage. If the system includes a smart diverter, the flow of fluid may automatically be redirected to a separate milk collection bottle once the colostrum collector reaches capacity or when higher-flow milk is detected. Throughout the session, data may be recorded and stored for later review. This data may help caregivers or clinicians assess postpartum recovery, monitor milk output trends, or refine feeding strategies for newborns. In some cases, the system may generate alerts if flow is not detected within a certain timeframe or if the collector is at risk of overflowing. These alerts help to ensure timely response, minimize waste, and maintain the hygienic integrity of the collected colostrum.

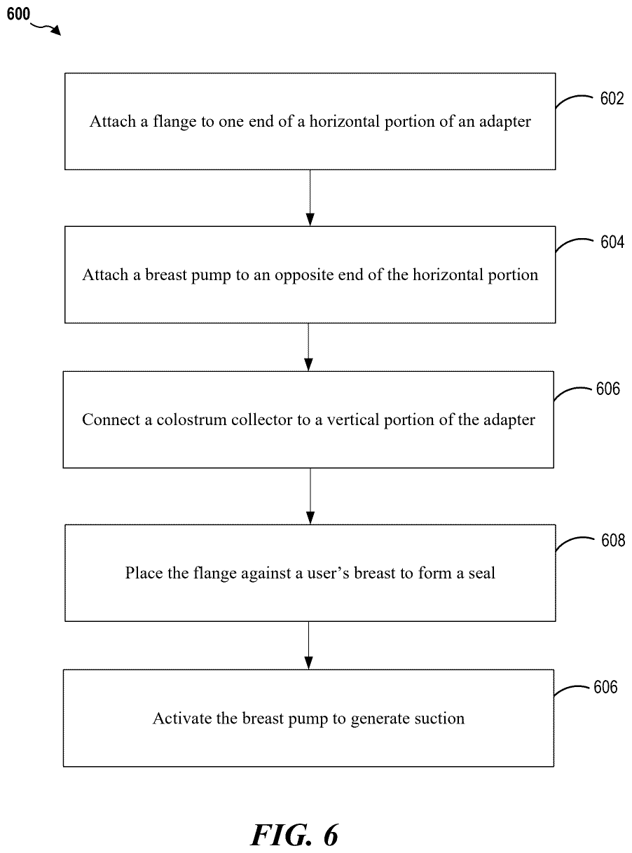

FIG. 6 illustrates a flow diagram for a process 600 of using a colostrum collection system, in accordance with various aspects of the present disclosure. The process 600 may be performed by a user with one or more components of a colostrum collection system, such as a colostrum collection system 150 or 300 described with reference to FIGS. 1A, 1B, 3A, or 3B. The process 600 begins at block 602 by a user attaching a flange to one end of a horizontal portion of an adapter. At block 604, the user attaches a breast pump to an opposite end of the horizontal portion. At block 606, the user connects a colostrum collector to a vertical portion of the adapter. At block 608, the user places the flange against a user's breast to form a seal. At block 610, the user activates the breast pump to generate suction, such that colostrum is expressed from the user's breast into the flange, and the expressed colostrum is diverted through the adapter into the colostrum collector via the vertical portion.

In some examples, the process 600 also includes assembling a colostrum collection system using a manual breast pump, a pump adapter, and a pre-sterilized collector. In such examples, the user assembles the manual breast pump, setting the flange aside temporarily. The pump adapter is then inserted into the top opening of the manual pump until it fits securely. Next, the flange is attached to the opposite end of the pump adapter and gently pushed into place to ensure a secure seal. A pre-sterilized colostrum collector is then removed from its packaging. The user removes the keep-clean cap and any attached suction or feeding tip from the collector and sets them aside. Finally, the lower end of the pump adapter is inserted into the opening of the colostrum collector, and the user presses upward on the collector to achieve a snug and secure fit.

In some examples, the adapter is inserted into the opening of the colostrum collector, rather than the collector being inserted into the adapter. Additionally, or alternatively. In other examples, the horizontal portion of the adapter may include an annular airflow path, as opposed to a single central duct. The annular airflow path may form a ring-shaped or circumferential passage that surrounds an internal diverter structure. This configuration enables continuous and evenly distributed airflow between the pump and the flange, reducing suction loss and promoting more consistent pressure generation across the expression system.

In certain examples, the vertical port of the adapter may be dimensioned to preclude direct connection with a standard syringe. This may be achieved by selecting an outer diameter that exceeds typical syringe inlet dimensions (e.g., greater than 5 mm), thereby preventing unintended withdrawal, contamination, or misuse in clinical environments. This structural limitation improves hygiene and ensures the colostrum remains within the sealed, designated collection pathway. Of course, in other examples, a syringe may be attached to the adapter to extract colostrum.

In some examples, the diverter may include a central channel with a sloped or curved inner wall, rather than a flat or perpendicular surface. This geometry is configured to direct colostrum into the vertical port with minimal turbulence, reducing adhesion to internal surfaces and promoting efficient, laminar flow into the collector. Additionally, in some examples, the adapter may include a dual-diverter configuration. In such examples, the adapter may be equipped with multiple diverter structures or actively controlled valves that direct fluid into separate collection pathways or compartments. This may allow colostrum to be divided for quality control, tracked independently for each breast, or routed to different storage containers based on user or clinical requirements.

As used herein, the term “determining” encompasses a wide variety of actions. For example, “determining” may include calculating, computing, processing, deriving, investigating, looking up (e.g., looking up in a table, a database or another data structure), ascertaining and the like. Additionally, “determining” may include receiving (e.g., receiving information), accessing (e.g., accessing data in a memory) and the like. Furthermore, “determining” may include resolving, selecting, choosing, establishing, and the like.

As used herein, a phrase referring to “at least one of” a list of items refers to any combination of those items, including single members. As an example, “at least one of: a, b, or c” is intended to cover: a, b, c, a-b, a-c, b-c, and a-b-c.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

It is to be understood that the claims are not limited to the precise configuration and components illustrated above. Various modifications, changes, and variations may be made in the arrangement, operation, and details of the methods and apparatus described above without departing from the scope of the claims.

What is claimed is:

1. A colostrum collection device, comprising:
a lower funnel-shaped component;
an upper bulb component integrated with the lower funnel-shaped component; and
a cap for sealing the lower funnel-shaped component, the colostrum collection device being attachable to a T-shaped adapter.

2. The colostrum collection device of claim 1, wherein the upper bulb component serves as a feeding mechanism.

3. The colostrum collection device of claim 1, wherein the T-shaped adapter includes a horizontal portion and a vertical portion that is perpendicular to the horizontal portion.

4. The colostrum collection device of claim 3, wherein the horizontal portion includes a first opening to attach to a flange and a second opening to attach to a breast pump.

5. The colostrum collection device of claim 4, wherein the first opening is wider than the second opening.

6. The colostrum collection device of claim 3, wherein the lower funnel-shaped component attaches to the vertical portion.

7. The colostrum collection device of claim 3, wherein the vertical portion has a tapered end.

8. The colostrum collection device of claim 3, wherein the T-shaped adapter includes a diverter for diverting colostrum to the vertical portion.

9. The colostrum collection device of claim 3, wherein the breast pump is a manual breast pump.

10. A colostrum collection system, comprising:
a breast pump;
a flange configured to form a seal against a user's breast;
a T-shaped adapter comprising a horizontal portion and a perpendicular vertical portion;
a colostrum collector attachable to the vertical portion of the adapter, the collector comprising a lower funnel-shaped body, an upper bulb component, and a tethered cap.

11. The colostrum collection system of claim 10, wherein the T-shaped adapter includes a diverter for diverting colostrum to the vertical portion.

12. The colostrum collection system of claim 10, wherein the colostrum collection device, comprises:
a lower funnel-shaped component; and
an upper bulb component integrated with the lower funnel-shaped component.

13. The colostrum collection system of claim 10, wherein the breast pump is a manual breast pump.

14. The colostrum collection device of claim 10, wherein the horizontal portion includes a first opening to attach to the flange and a second opening to attach to the breast pump.

15. The colostrum collection device of claim 14, wherein the first opening is wider than the second opening.

16. A method for collecting colostrum using a colostrum collection device, comprising:
attaching a flange to one end of a horizontal portion of an adapter;
attaching a breast pump to an opposite end of the horizontal portion;
connecting a colostrum collector to a vertical portion of the adapter;
placing the flange against a user's breast to form a seal; and
activating the breast pump to generate suction, such that colostrum is expressed from the user's breast into the flange, and the expressed colostrum is diverted through the adapter into the colostrum collector via the vertical portion.

17. The method of claim 16, further comprising attaching a suction tip to the upper bulb.

18. The method of claim 16, further comprising using an upper bulb component of the colostrum collector to feed colostrum directly to an infant.

19. The method of claim 16, wherein the adapter includes a diverter for diverting colostrum to the vertical portion of the adapter and directing milk to a separate container.

20. The method of claim 16, wherein the breast pump is a manual breast pump.
